(19) United States
(12) Patent Application Publication
(10) Pub. No.: US 2025/0320492 A1
(43) Pub. Date: Oct. 16, 2025

(54) FLEXIBLE RNA SCAFFOLDING FOR REPROGRAMMABLE COMBINATORIAL RNAi ADMINISTRATION

(71) Applicant: Speratum Biopharma, Inc., Dover, DE (US)

(72) Inventors: Christian Roberto Marin-Muller, Dover, DE (US); Osvaldo Vega Martinez, Dover, DE (US); Juan Carlos Valverde-Hernandez, Dover, DE (US)

(73) Assignee: Speratum Biopharma, Inc., Dover, DE (US)

(21) Appl. No.: 18/022,263

(22) PCT Filed: Aug. 20, 2021
(86) PCT No.: PCT/US2021/047006

§ 371 Date: Feb. 20, 2023

Related U.S. Application Data
(60) Provisional application No. 63/067,966, filed on Aug. 20, 2020.

(51) Int. Cl. C12N 15/11

(52) U.S. Cl. CPC C12N 15/111; C12N 2310/141; C12N 2310/3519; C12N 2310/531

(57) ABSTRACT

Provided is a method for administering a single RNAi molecule or a combinatorial RNAi molecule via a polycistronic RNAi scaffold, where the polycistronic RNAi scaffold comprises one or more arms disposed on the polycistronic RNAi scaffold, wherein each of the one or more arms is an individual RNAi substrate, where at least one of the one or more arms comprise one or more of: a dicer independent loop sequence, a dicer independent loop-closing sequence, and a corresponding complement sequence. Further, at least one of the one or more arms may further comprise one or more of: an Ago2 cleavage sequence, a corresponding seed sequence, and a MID domain tight interaction.

### CLAIM OF PRIORITY

This application claims priority from U.S. Provisional Patent Application No. 63/067,966, filed on Aug. 20, 2020, the contents of which are incorporated herein by reference.

### SEQUENCE LISTING

The instant application contains a Sequence Listing which has been submitted electronically in ASCII format and is hereby incorporated by reference in its entirety. Said ASCII copy, created on Sep. 29, 2023, is named 1082_003PCT_US_SL.txt and is 15,599 bytes in size.

### FIELD OF THE INVENTION

The invention is related generally to the field of RNAi sequences. More particularly, the invention relates to apparatuses, methods, and systems for RNAi administration via flexible RNA scaffolding.

### BACKGROUND

Use of ribonucleic acid (RNA), and in particular, RNA-interference (RNAi) has long been proposed and explored for therapeutic purposes. Utilization of RNAi opened up treatments of previously untreatable diseases.

Certain diseases, such as cancer, are multimodal and heterogeneous; and others, such as viruses, have potential target sites that may mutate. Thus, it would be desirable to use multiple small-interfering RNAs (siRNA) or MicroRNAs (miRNA) molecules that can target either the same target in multiple places, different targets individually, or different targets simultaneously.

Moreover, since a potent silencing effect may not be sufficiently obtained through the use of just one siRNA or miRNA, because complex diseases require many molecular targets to be silenced in order to achieve a clinically relevant effect, many studies therefore utilize siRNA pools, where multiple siRNA molecules are used simultaneously. This results in several siRNA-based therapies utilizing multiple siRNA molecules.

Further, RNAi molecules have some limitations that may be ameliorated when using combinations of RNAi molecules. Effects such as target site mutation, genetic variability and off target effects are some of these problems.

However, such therapies and pathways face difficulties in achieving proper molecular stoichiometries and specific quantities and ratios of each siRNA per dose. It would be desirable, therefore, to provide administration of combinations of RNAi molecules in a relatively straight-forward and easy format, such as conventional drug delivery systems.

### BRIEF DESCRIPTIONS OF THE DRAWINGS

FIGS. 1A-1C illustrate an embodiment of a non-pass mimic cluster platform for generating two RNAi molecules. FIG. 1 discloses SEQ ID NOS 1-3, 5, 4, 6-9, 11, 10, 12, 5, and 4, respectively, in order of appearance.

FIG. 2 illustrates an embodiment of the structure of a single arm of a scaffold, a non-canonical microRNA mimic. Figure discloses SEQ ID NO: 13.

FIG. 3 is a graph illustrating an embodiment of a luciferase assay demonstrating the efficacy of a miR-198 containing arm in vitro.

FIG. 4 is a graph illustrating an embodiment of a molecular target regulation demonstrating the efficacy of a miR-198 containing arm in vitro.

FIG. 5 is a graph illustrating an embodiment of a molecular target regulation demonstrating the efficacy of a miR-34 containing arm in vitro.

FIG. 6 is a graph illustrating an embodiment of a molecular target regulation demonstrating the efficacy of a miR-29b containing arm in vitro.

FIG. 7 is a graph illustrating an embodiment of administration of single miR-198 containing arms in vitro in six different cancer cell lines.

FIG. 8 is a graph illustrating an embodiment of a use of a single arm delivered in vivo.

FIGS. 9A-F are graphs illustrating an embodiment of toxicity profiles using single arm.

### DETAILED DESCRIPTION

Provided herein is a reprogrammable, polycistronic RNAi scaffold for administration of a single type of RNAi, as well as multiple combinations of different RNAi sequences.

The RNAi polycistronic scaffold may enable the administration of combinations of RNAi molecules administered as a single RNA molecule with multiple arms, where each arm may be an individual RNAi substrate, and may be any oligonucleotide molecule. In some embodiments, the arms of the scaffold are RNAi substrates that function via non-canonical processing pathways. This scaffold permits use as a miRNA or siRNA administration platform, and may be used in vitro or in vivo. It may be delivered using conventional drug delivery systems and may be chemically modified. However, in an embodiment, administration is not limited to conventional drug delivery systems.

In an embodiment where combinatorial RNAi molecules are needed for administration, the modular nature of this scaffold makes stoichiometric ratio control possible. That is, the RNAi scaffold may act like an RNAi cluster with multiple effector arms. In one embodiment, each arm may be different siRNA or miRNA molecules. In another embodiment, one or more of the scaffold arms may be the same molecule, and, in yet another embodiment, each of the arms may be administered independently. Thus, the proposed scaffold may compile a series of motifs that may make an efficient RNAi maturation process possible. In an embodiment, it may be adapted to be both Dicer-dependent or Dicer-independent.

Thus, in accordance with an embodiment, any miRNA or siRNA sequence may be incorporated into this scaffold. In a therapeutic setting, the scaffold and its arms may function as a prodrug, requiring further intracellular processing.

The scaffold may include a number of benefits. One such benefit may be the ability to deliver multiple miRNA or siRNA sequences by achieving an adequate stoichiometric administration of a specific number of each respective sequence (for example two molecules of siRNA sequence A and one molecule of siRNA sequence B per each administered scaffold). The sequence for each arm may be the same, or may vary by at least one nucleotide. Another such benefit may be the ability for the scaffold to be an siRNA/miRNA mimic hybrid, with each arm as a different RNAi substrate. These separate arms may be modified or unmodified siRNA or miRNA mimic structures. A further benefit may be that the scaffold and its arms may be generated in cell free systems, simplifying the molecule isolation and purification process. Alternatively, another benefit may be that the scaffold or its arms may be expressed using an expression vector, and will maintain its integrity and functional benefits. A further benefit may be that in comparison with combinatorial duplex administrations, fewer production and characterization steps may be required (for example, due to a need to only generate a single chain molecule).

Thus, while expression vectors may traditionally be used for introducing pri-RNAi into a cell, such an approach may introduce an abnormal strain to the cell. However, in an alternate embodiment, expression vectors may be used for introducing pri-RNAi into a cell. Further, in an embodiment, motif selection combinations include nucleus localization motifs, which may not have previously been incorporated to pri-RNAi designs. In one embodiment, this specific motif may be relevant to RNAi processing, since Drosha processing steps may take place within the nucleus.

In accordance with an embodiment, motifs may be compiled to promote efficient Drosha processing, and may be adjusted for Dicer-dependent and independent processing routes. In an embodiment, this may be useful in diseases where Dicer molecular machinery may be downregulated, such as cancer cases. In an embodiment, due to the molecule being administered as an isolated RNA, there may be no dependence on transcription machinery, resulting in non-interference with normal cellular transcription processes.

In some embodiments, the design may be generated in cell-free systems, thereby simplifying the molecule isolation process. In contrast with combinatorial shRNA or RNAi duplex administrations, the present invention may result in less production and characterization steps, since the present invention may require generation of only a single molecule. However, in alternate embodiments, the present invention may embrace qualities of combinatorial shRNA or RNAi.

In accordance with various embodiments, bi-cistronic or polycistronic structures may be used, and may contain siRNA, miRNA or other oligonucleotide structures in each arm.

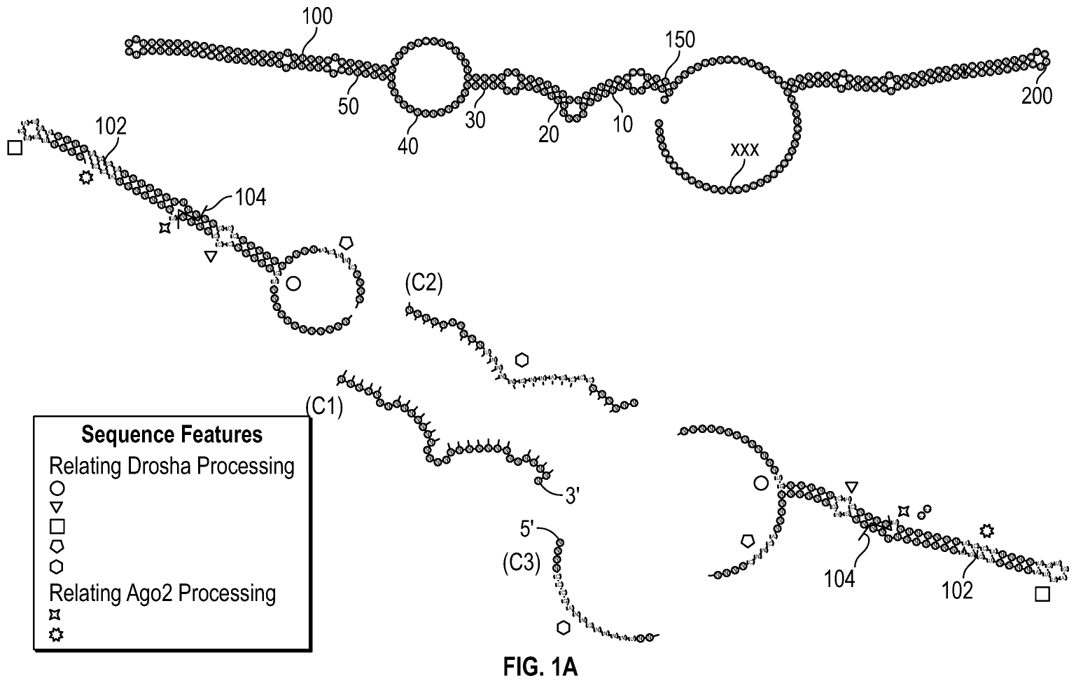

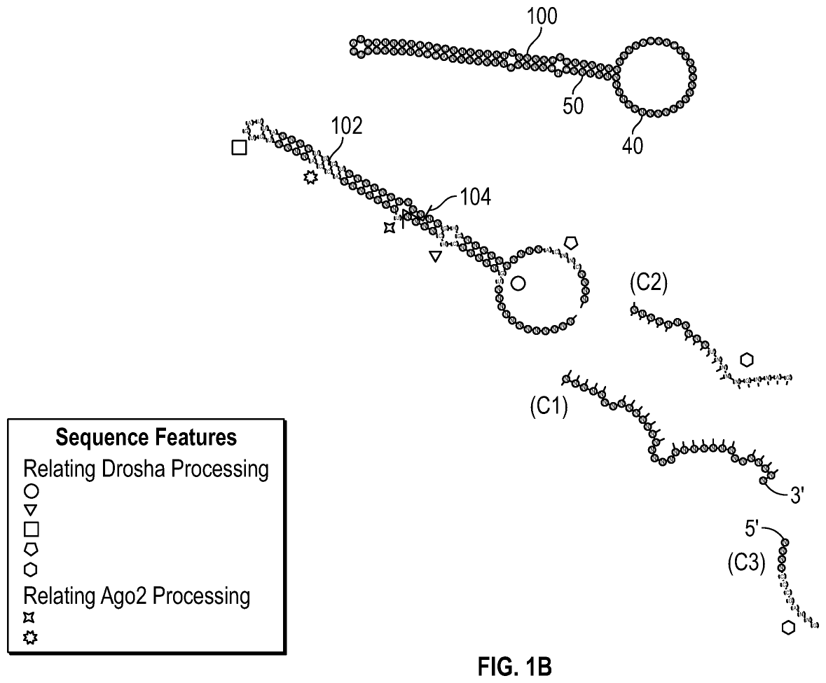

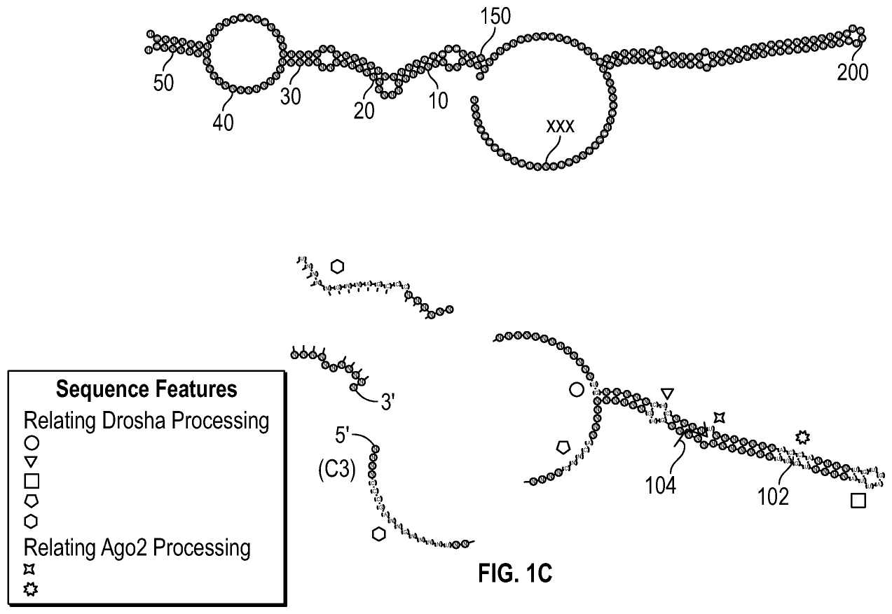

FIGS. 1A-1C illustrate an embodiment of the non-pass mimic cluster platform for generating two RNAi molecules. FIG. 1A is a view of the entire platform, with FIGS. 1B-1C representing views of portions thereof. As illustrated, (A) Accounts to the whole structure, (B) accounts to the first arm with a specific RNAi sequence, (C) accounts to the linker regions which includes a nuclear localization motif and (D) is the second arm with a specific RNAi sequence.

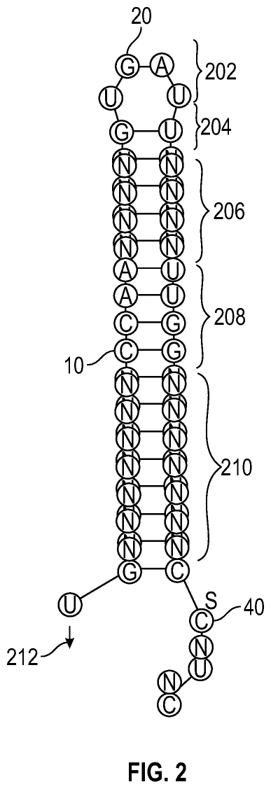

FIG. 2 illustrates an embodiment of a structure of a single arm of a scaffold, a non-canonical microRNA mimic. In an embodiment, the structure includes a dicer independent loop sequence 202, a dicer independent loop-closing sequence 204, a corresponding complement sequence 206, an Ago2 cleavage sequence 208, a corresponding seed sequence 210, and a MID domain tight interaction 212. However, in another embodiment, the structure may include any number or combination of the aforementioned sequences or interactions.

In order to recognize the microprocessor complex, a stem-loop structure may be used. In certain embodiments, the structure may be a highly complementary stem, flanked by unpaired segments corresponding to the Drosha cleavage site and thirteen unpaired-base pairs, starting at the 13^{th} upstream position from the 5′ Drosha cleavage site and at the 11^{th} downstream position from the 3′ Drosha cleavage site. This is shown in FIG. 1. Referring to FIGS. 1A-1C, the non-pass mimic cluster may comprise one or more Ago2 cut sites 102 and one or more Drosha cut sites 104. FIGS. 1-2 may be labeled for nucleotide positioning along the structure (for example, 10, 20, 30, 40, 50, 100, 150, and 200).

In some embodiments, the sequence 5′-UG-3′ may be at the 14^{th} upstream from the 5′ Drosha cleavage site for Drosha recognition of the stem-loop. The sequence 5′-UGUG-3′ may be, in some embodiments, at 17-20 specific position for an increased stem-loop structure recognition by the microprocessor complex. In an embodiment, in order to increase silencing activity by the miRNA, the hairpin loop may consist of the sequence 5′-UGAU-3′. In some embodiments, the sequence 5′-ACNNC-3′ may be at the 16^{th} downstream position from the 3′ Drosha cleavage site in order to promote stem-loop structure recognition by Drosha. In an embodiment, in order to promote the effective recognition of the RNA stem-loop by Drosha, the sequence 5′-GHG-3′ may, in certain embodiments, be at the 3^{rd} downstream position from the 3′ Drosha cleavage site, where “H” nucleotide may be unpaired. In an alternate embodiment, any suitable sequence may be disposed at any relevant corresponding position.

In an embodiment, in order to promote a stable overall molecular structure, and to enable desirable molecular interactions, the invention may utilize linker sequences designed to: (a) stabilize the miRNA stem-loop structures, such that a highly complementary 35 to 45 nucleotides long sequence must be at the ends of the miRNA precursor sequences; (b) in order to reduce the potential immunogenicity caused by highly complementary RNA molecules, placing three internal loops at the specific positions 5, 14, and 25 of the molecule; (c) in order to add the motif described herein, below, a 15 to 25 nucleotides long linker sequence is added at the 3′ end of the second miRNA precursor sequence; and/or (d) in order to promote the nuclear localization of the cluster structure, specific sequence 5′-UNNNNGNNAGCCC-3′ (SEQ ID NO: 14) is added at both ends.

In embodiments, the production process of the molecule involves in vitro transcription. In a cell free system embodiment, the isolation procedure may be much simpler compared to expression vector production. In an embodiment, if combinatorial RNAi duplexes need to be administered, fewer QC/QA processes may be required. In an embodiment, every duplex production process may require the synthesis of two ssRNA molecules. Each molecule may need to be compliant with QC/QA standards. After this, the ssRNA (RNAi duplex) may go through a QC/QA process.

Thus, in embodiments where 3 RNAi molecules need to be administered, the traditional method utilizes at least 9 QC/QA rounds, whereas, in accordance with this invention, only one round may be needed. However, in an alternate embodiment, the present invention may include any number of QC/QA rounds. In an embodiment, due to the scaffold, since the RNAi sequences are attached in a single molecule, stoichiometric ratios may be guaranteed in a multi-component therapeutic. In an embodiment, the efficacy of the molecules for in vivo use may therefore greatly improve as applied to the interaction with the polymer described herein (for example, making this a potentially more useful technology for in vitro and in vivo use).

In various embodiments, the invention may be used for directed modulation of any RNA molecule in vitro or in vivo. In accordance with the embodiments set forth herein, the invention may be utilized in a reprogrammable manner to incorporate any natural or designed RNAi-inducing sequence, and combinatorial RNAi ratios may be modulated in a stoichiometric manner. Therefore, the invention may be used to administer any miRNA, siRNA, or miRNA/siRNA hybrid molecules, either through including single specific seed sequences, or fully designed and engineered RNAi-inducing molecules either as a cluster or as individual arms.

Regarding cell culture and transfection, in an embodiment, human cancer cell lines may be grown in vitro in the corresponding culture medium at 5% CO₂ and 37° C. An NPM platform may be used for transient transfection of miRNA-198, miRNA-34a, miRNA-29b, miR-608 (NPM198/34a/29b/608) or miR-control (NPM-NC) in human cancer cells. In general, 1×10⁶ cells may be seeded on each well of a 6-well plate in OptiMEM medium and transient transfected using Lipofectamine3000 (for example, per manufacturer instruction).

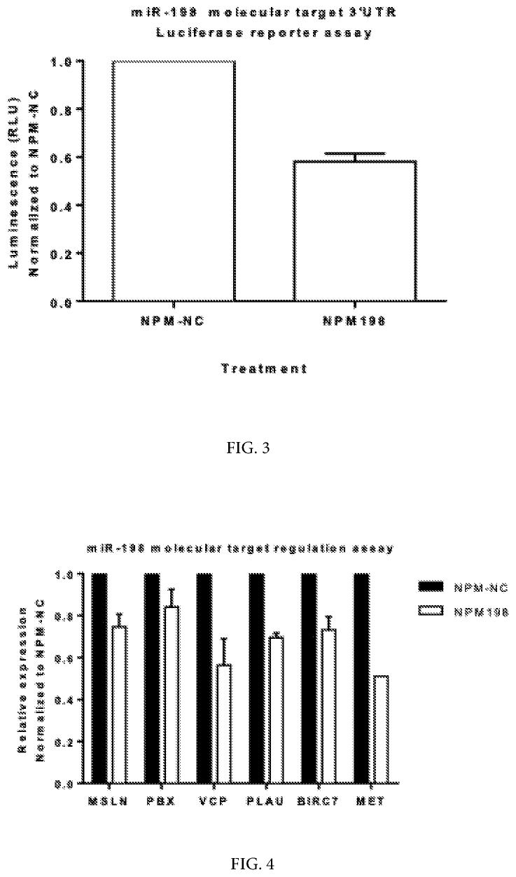

Regarding luciferase reporter assay, in an embodiment, fractions of the 3′UTR of a target gene may be cloned into the pmirGLO vector (Promega) and transient transfected into human cancer cell line using Lipofectamine3000 (for example, per manufacturer instruction). Then, 5×10⁴ cells may be plated into triplicates wells of a 96-well plate and retransfected with 30 pmol of any NPM198/34a/29b/608 or miR-control using Lipofectamine3000 (for example, per manufacturer instruction). After 24 hours, cell luciferase activity may be measured using the Dual-Glo® Luciferase Assay System Kit (Promega) (for example, per manufacturer instruction). FIG. 3 is a graph illustrating an embodiment of a luciferase assay demonstrating the efficacy of a miR-198 containing arm in vitro.

Regarding in vitro cell proliferation assay, in an embodiment, to determine cellular proliferation, 5×10³ previously transfected cells may be seeded into quintuplicates wells of a 96-well plate. Cell proliferation may be assessed after 48 hours using the Cell Titer 96® Aqueous One Solution Cell Proliferation MTS Assay (PROMEGA) (for example, per manufacturer instructions). The colorimetric assay may be read at 490 nm in a microplate reader (BioTek Synergy™ H1, Winooski, VT).

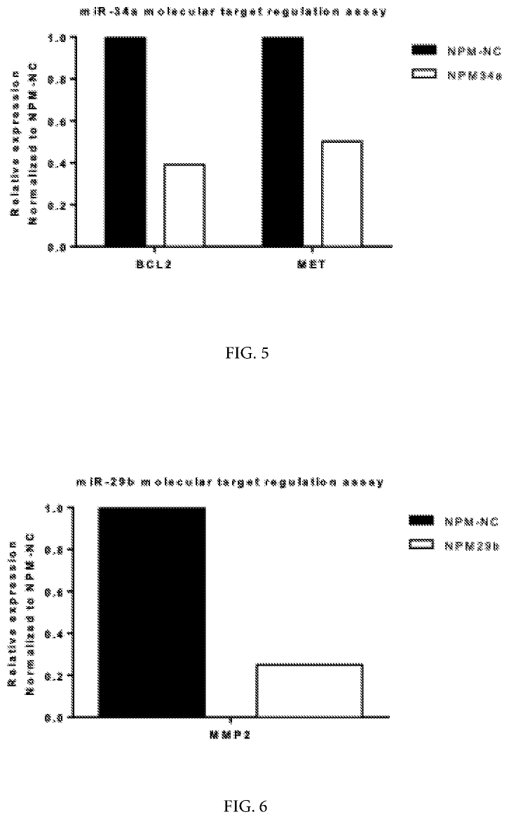

FIG. 4 is a graph illustrating an embodiment of a molecular target regulation demonstrating the efficacy of a miR-198 containing arm in vitro. FIG. 5 is a graph illustrating an embodiment of a molecular target regulation demonstrating the efficacy of a miR-34 containing arm in vitro. FIG. 6 is a graph illustrating an embodiment of a molecular target regulation demonstrating the efficacy of a miR-29b containing arm in vitro.

Regarding a subcutaneous xenograft mice model, in an embodiment, animal procedures may be conducted under guidelines approved by the Costa Rican Institutional Animal Care and Use Committee (CICUA). Human cancer cells (1.5×10⁶) in 200 μL of a 1:1 mixture of culture medium and matrigel may be inoculated into the flanks of 4-week-old female nude mice (NCI-Charles River) as described. After 7 days, upon tumor establishment, tumors volume may be measured and, mice may be randomized into groups with comparable initial tumor volume means. Mice may be weighed and tumor size may be measured using digital calipers every treatment day before injection. Tumor volume may be determined with the following formula:

$$Tumor volume ( mm 3 ) = [ length ( mm ) ] \times {[ width ( mm ) ]}^{2} \times 0.52 .$$

In an embodiment, a dosage of 2 mg/kg of either NPM198/34a/29b/608 or NPM-NC may be prepared for injection using a polymeric nanoparticle formulation protocol as described. In an embodiment, day 0 may correspond to the day of the randomization into groups and the start of the treatment injections. Mice may be injected a total of six times, on days 0, 2, 4, 7, 9 and 11. The mice may be euthanized when the largest tumor reaches a diameter of 2000 mm³ or 3 days after treatments may be completed. At this point, mice may be evaluated macroscopically for the presence of tumor/metastases. In an embodiment, tumor nodules may be explanted, weighed, and stored in RNALater solution at −80° for subsequent analysis.

Regarding orthotopic xenograft mice model, in an embodiment, animal procedures may be conducted under guidelines approved by the Costa Rican Institutional Animal Care and Use Committee (CICUA). Human cancer cells (1×10⁶) in 50 μL of a 2:3 mixture of culture medium and matrigel may be inoculated into the corresponding organ of 4-week-old female nude mice (NCI-Charles River) as described. After 21 days, upon tumor establishment, tumors volume may be measured and, mice may be randomized into groups with comparable initial tumor volume means. Mice may be weighed and tumor size may be measured using an ultrasound machine before treatment assignation and after last treatment dosification. In an embodiment, tumor volume was determined with the following formula:

$$Tumor volume ( mm 3 ) = [ length ( mm ) ] \times {[ width ( mm ) ]}^{2} \times 0.52 .$$

A dosage of 2 mg/kg of either NPM198/34a/29b/608 or NPM-NC may be prepared for injection using a polymeric nanoparticle formulation protocol as described. Day 0 may correspond to the day of the randomization into groups and the start of the treatment injections. Mice may be injected a total of twelve times. All mice may be euthanized when the largest tumor reached a diameter of 2000 mm³ or 3 days after treatments were completed. At this point, mice may be evaluated macroscopically for the presence of tumor/metastases. In an embodiment, tumor nodules may be explanted, weighed, and stored in RNALater solution at −80° for subsequent analysis.

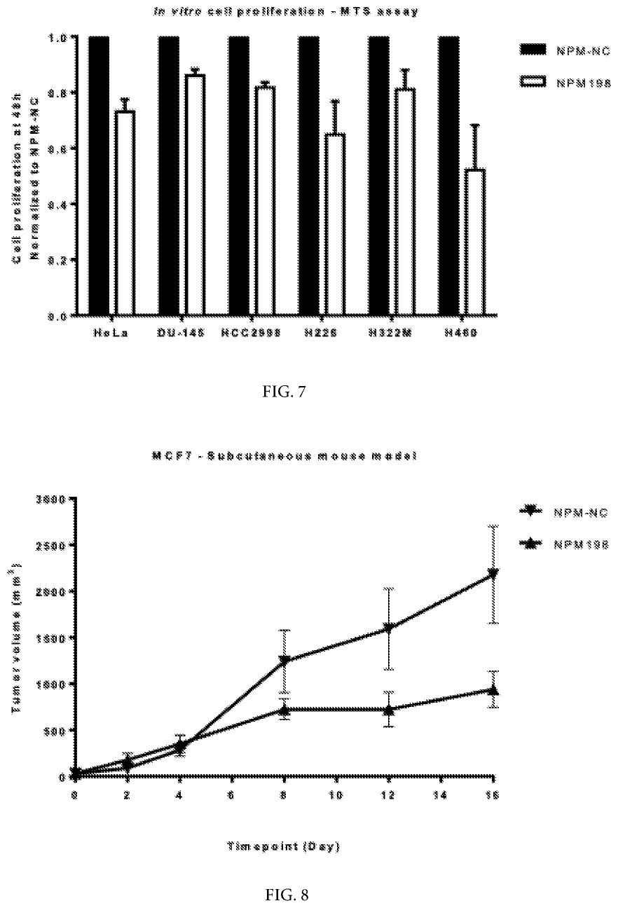

FIG. 7 is a graph illustrating an embodiment of administration of single miR-198 containing arms in vitro in six different cancer cell lines. FIG. 8 is a graph illustrating an embodiment of use of a single arm delivered in vivo.

Regarding an in vivo mouse safety study, in an embodiment, animal procedures may be conducted under guidelines approved by the Costa Rican Institutional Animal Care and Use Committee (CICUA). Four-week-old female CD-1 mice (NCI-Charles River) may be dosed 3 times per week for 4 weeks with polymeric nanoparticles containing 10 mg/kg of NPM198. Saline solution may be used as a negative control. In an embodiment, two days after the last dosification, mice may be euthanized, and blood and organs may be collected.

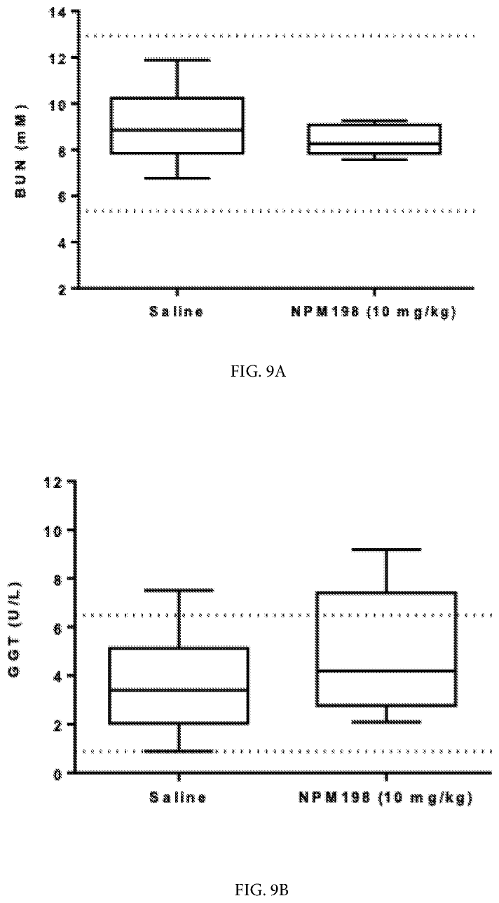

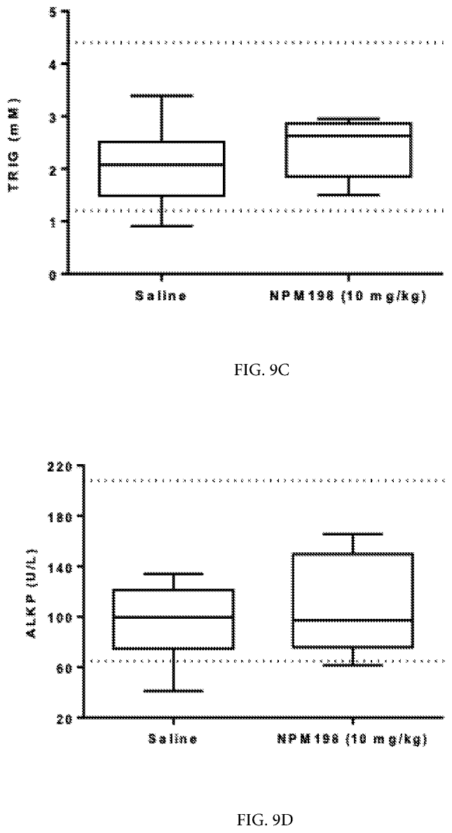

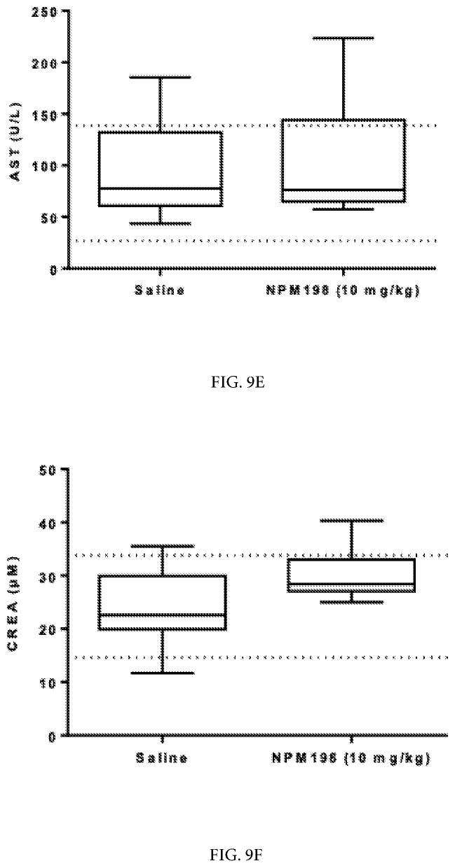

Mice may be observed along the dosification study for health-related events and at the end of the study, blood chemistry and hematology analysis may be done to determine preliminary toxicity related to nanoparticles containing NPM198 exposure. FIGS. 9A-F are graphs illustrating an embodiment of a toxicity profile using single arm.

Regarding Biomolecule extraction, in an embodiment, cells may be collected 24 hours after transfection. Total RNA may be extracted from the transfected cell lines using mirVana™ RNA Purification Kit per manufacturer instruction. The concentration and purity of the RNA extracts may be performed using a Nanodrop device. In an embodiment, the integrity of the RNA may be evaluated through agarose electrophoresis.

Regarding mRNA relative expression analysis, in an embodiment, for each molecular target, primers may be designed and synthetized according to standard qPCR primer design rules. A PPIA gene may be selected as a reference gene based on scientific reports.

Regarding any of the aforementioned procedures, in various embodiments, the times, steps, volumes, measurements, structures, or any other components of the procedures, may be replaced with suitable substitutes. Thus, for the purposes of this disclosure, the aforementioned procedures are non-limiting.

In accordance with various embodiments described herein, the invention may be administered as an RNAi mimic, or in an expression vector, such as a virus, plasmid or any other suitable vector. The invention may further be used in combination with LNP, polymeric nanoparticles, aptamer associated delivery methods, antibody associated delivery methods, affimer associated delivery methods, nanoparticles, or any other suitable methods.

In accordance with embodiments herein, the platform may be used for administering any RNAi sequence, such as a biological or synthetic one, and with any standard of care. The platform may be used with Dicer processing, but need not, and may be administered with any RNAi molecule, whether in an individual or combinatorial manner. The platform may be administered as either linear or circular RNA. The arms may be used independently or as a cluster.

The invention of the present invention may be a method for administering a single RNAi molecule via a polycistronic RNAi scaffold, where the polycistronic RNAi scaffold comprises one or more arms disposed on the polycistronic RNAi scaffold, where each of the one or more arms is an individual RNAi substrate, where at least one of the one or more arms comprises a dicer independent loop sequence, a dicer independent loop-closing sequence, and a corresponding complement sequence. However, in an alternate embodiment, each of the one or more arms may not be an individual RNAi substrate (for example, one arm may be an individual RNAi substrate and a second arm may not be an individual RNAi substrate). In another embodiment, at least one of the one or more arms may comprise one or more of: a dicer independent loop sequence, a dicer independent loop-closing sequence, and a corresponding complement sequence. In a further embodiment, at least one of the one or more arms may further comprise one or more of: an Ago2 cleavage sequence, a corresponding seed sequence, and a MID domain tight interaction.

In an embodiment, at least one of the one or more arms may comprise one or more unpaired segments corresponding to one or more Drosha cleavage sites and thirteen unpaired-base pairs, starting at a 13^{th} upstream position from a 5′ Drosha cleavage site and at a 11^{th} downstream position from a 3′ Drosha cleavage site. However, in an embodiment, at least one of the one or more arms may comprise one or more unpaired segments corresponding to one or more Drosha cleavage sites and any number of unpaired-base pairs, starting at any suitable upstream position from any suitably positioned Drosha cleavage site and at any suitable downstream position from any suitable Drosha cleavage site.

In an embodiment, a sequence 5′-UG-3′ may be disposed 14^{th} upstream from a 5′ Drosha cleavage site for Drosha recognition of a stem-loop. However, in another embodiment, a sequence 5′-UG-3′ may be disposed any suitable position upstream from any Drosha cleavage site for Drosha recognition of a stem-loop. In an embodiment, at least one of the one or more arms may further comprise a hairpin loop, the hairpin loop may include a sequence 5′-UGAU-3′. However, in another embodiment, the hairpin loop may include any comparable sequence or any suitable sequence. In an embodiment, at least one of the one or more arms may further include one or more linker sequences, the one or more linker sequences may be configured to stabilize a miRNA stem-loop structure, such that a highly complementary 35 to 45 nucleotides long sequence may be disposed at an end of a miRNA precursor sequence. However, in another embodiment, at least one of the one or more arms may further include one or more linker sequences, the one or more linker sequences may be configured to stabilize a miRNA stem-loop structure, such that any suitable sequence (for example, a sequence of any suitable nucleotide length) may be disposed at an end, or at any position, on a miRNA precursor sequence. In a further alternate embodiment, at least one of the one or more arms may further include one or more linker sequences, the one or more linker sequences may be configured to stabilize any relevant structure.

In an embodiment, the individual RNAi substrate may be an oligonucleotide molecule. However, the individual RNAi substrate may have a polynucleotide whose molecules contain any number of nucleotides. In an embodiment, at least one of the one or more arms of the scaffold may be RNAi substrates that function via a non-canonical processing pathway. In an alternate embodiment, at least one of the one or more arms of the scaffold may be RNAi substrates that function via a canonical processing pathway. In an embodiment, the administration of the single RNAi molecule may occur in vitro or in vivo.

In an embodiment, the invention of the present disclosure may be a method for administering a combinatorial RNAi molecule via a polycistronic RNAi scaffold, where the polycistronic RNAi scaffold comprises one or more arms disposed on the polycistronic RNAi scaffold, where at least one of the one or more arms comprise a dicer independent loop sequence, a dicer independent loop-closing sequence, and a corresponding complement sequence. In another embodiment, at least one of the one or more arms may comprise one or more of: a dicer independent loop sequence, a dicer independent loop-closing sequence, and a corresponding complement sequence. In a further embodiment, at least one of the one or more arms may further comprise one or more of: an Ago2 cleavage sequence, a corresponding seed sequence, and a MID domain tight interaction.

In an embodiment, at least one of the one or more arms may further comprise one or more unpaired segments corresponding to one or more Drosha cleavage sites and thirteen unpaired-base pairs, starting at a 13^{th} upstream position from a 5′ Drosha cleavage site and at a 11^{th} downstream position from a 3′ Drosha cleavage site. However, in another embodiment, at least one of the one or more arms may further comprise one or more unpaired segments corresponding to one or more Drosha cleavage sites and any number of unpaired-base pairs, starting at any suitable upstream position from any Drosha cleavage site and at any downstream position from any Drosha cleavage site. In an embodiment, a sequence 5′-UG-3′ may be disposed 14^{th} upstream from a 5′ Drosha cleavage site for Drosha recognition of a stem-loop. However, in an alternate embodiment, any sequence may be disposed at any upstream position from any Drosha cleavage site for Drosha recognition of any suitable structure (for example, a stem-loop). In an embodiment, at least one of the one or more arms may comprise a hairpin loop, the hairpin loop may comprise a sequence 5′-UGAU-3′. However, in another embodiment, at least one of the one or more arms may comprise any suitable structure, the suitable structure comprising a sequence 5′-UGAU-3′. In a further alternate embodiment, at least one of the one or more arms may comprise a hairpin loop, the hairpin loop comprising any sequence.

In an embodiment, at least one of the one or more arms may further comprise one or more linker sequences, the one or more linker sequences may be configured to stabilize a miRNA stem-loop structure, such that a highly complementary 35 to 45 nucleotides long sequence is disposed at a end of a miRNA precursor sequence. However, the linker sequence may be configured for any purpose. Further, the linker sequence may stabilize any structure. Moreover, the linker sequence may stabilize a miRNA stem-loop structure, such that a sequence of any length may be disposed at any position on a miRNA precursor sequence (or any other suitable sequence). In an embodiment, each of the one or more arms may be: a different siRNA or miRNA molecule; the same siRNA or miRNA molecule; and/or administered independently. In an embodiment, the polycistronic RNAi scaffold may be configured to compile a series of motifs, the series of motifs may be configured to facilitate an efficient RNAi maturation process. However, in an alternate embodiment, the polycistronic RNAi scaffold may be configured to compile any suitable structure. In another embodiment, the series of motifs may be configured to facilitate any suitable RNAi process.

While this invention has been described in conjunction with the embodiments outlined above, many alternatives, modifications and variations will be apparent to those skilled in the art upon reading the foregoing disclosure. Accordingly, the embodiments of the invention, as set forth above, are intended to be illustrative, not limiting. Various changes may be made without departing from the spirit and scope of the invention.

What is claimed is:

1. A method for administering a single RNAi molecule via a polycistronic RNAi scaffold, wherein the polycistronic RNAi scaffold comprises:
one or more arms disposed on the polycistronic RNAi scaffold, wherein each of the one or more arms is an individual RNAi substrate, at least one of the one or more arms comprising:
a dicer independent loop sequence;
a dicer independent loop-closing sequence; and
a corresponding complement sequence.

2. The method of claim 1, wherein at least one of the one or more arms further comprises an Ago2 cleavage sequence, a corresponding seed sequence, and a MID domain tight interaction.

3. The method of claim 1, wherein at least one of the one or more arms further comprises one or more unpaired segments corresponding to one or more Drosha cleavage sites and thirteen unpaired-base pairs, starting at a 13^{th} upstream position from a 5′ Drosha cleavage site and at a 11^{th} downstream position from a 3′ Drosha cleavage site.

4. The method of claim 1, wherein a sequence 5′-UG-3′ is disposed 14^{th} upstream from a 5′ Drosha cleavage site for Drosha recognition of a stem-loop.

5. The method of claim 1, wherein at least one of the one or more arms further comprises a hairpin loop, the hairpin loop comprising a sequence 5′-UGAU-3′.

6. The method of claim 1, wherein at least one of the one or more arms further comprises one or more linker sequences, the one or more linker sequences configured to stabilize a miRNA stem-loop structure, such that a highly complementary 35 to 45 nucleotides long sequence is disposed at an end of a miRNA precursor sequence.

7. The method of claim 1, wherein the individual RNAi substrate is an oligonucleotide molecule.

8. The method of claim 1, wherein at least one of the one or more arms of the scaffold are RNAi substrates that function via a non-canonical processing pathway.

9. The method of claim 1, wherein the administration of the single RNAi molecule occurs in vitro.

10. The method of claim 1, wherein the administration of the single RNAi molecule occurs in vivo.

11. A method for administering a combinatorial RNAi molecule via a polycistronic RNAi scaffold, wherein the polycistronic RNAi scaffold comprises:
one or more arms disposed on the polycistronic RNAi scaffold, at least one of the one or more arms comprising:
a dicer independent loop sequence;
a dicer independent loop-closing sequence; and
a corresponding complement sequence.

12. The method of claim 11, wherein at least one of the one or more arms further comprises a dicer independent loop sequence, a dicer independent loop-closing sequence, a corresponding complement sequence, an Ago2 cleavage sequence, a corresponding seed sequence, and a MID domain tight interaction.

13. The method of claim 11, wherein at least one of the one or more arms further comprises one or more unpaired segments corresponding to one or more Drosha cleavage sites and a thirteen unpaired-base pairs, starting at a 13^{th} upstream position from a 5′ Drosha cleavage site and at a 11^{th} downstream position from a 3′ Drosha cleavage site.

14. The method of claim 11, wherein a sequence 5′-UG-3′ is disposed 14^{th} upstream from a 5′ Drosha cleavage site for Drosha recognition of a stem-loop.

15. The method of claim 11, wherein at least one of the one or more arms further comprises a hairpin loop, the hairpin loop comprising a sequence 5′-UGAU-3′.

16. The method of claim 11, wherein at least one of the one or more arms further comprises one or more linker sequences, the one or more linker sequences configured to stabilize a miRNA stem-loop structure, such that a highly complementary 35 to 45 nucleotides long sequence is disposed at a end of a miRNA precursor sequence.

17. The method of claim 11, wherein each of the one or more arms is a different siRNA or miRNA molecule.

18. The method of claim 11, wherein each of the one or more arms is the same siRNA or miRNA molecule.

19. The method of claim 11, wherein each of the one or more arms is administered independently.

20. The method of claim 11, wherein the polycistronic RNAi scaffold is configured to compile a series of motifs, the series of motifs configured to facilitate an efficient RNAi maturation process.


SEQUENCE LISTING (14 sequences)

SEQ ID NO: 1 - RNA, 264 nt, Artificial Sequence
  Description of Artificial Sequence Synthetic polynucleotide
  modified_base (2)..(4): a, c, u, g, unknown or other
  modified_base (6)..(51): a, c, u, g, unknown or other
  modified_base (55)..(58): a, c, u, g, unknown or other
  modified_base (60)..(66): a, c, u, g, unknown or other
  modified_base (71)..(74): a, c, u, g, unknown or other
  modified_base (83)..(86): a, c, u, g, unknown or other
  modified_base (91)..(102): a, c, u, g, unknown or other
  modified_base (106)..(115): a, c, u, g, unknown or other
  modified_base (118)..(119): a, c, u, g, unknown or other
  modified_base (121)..(142): a, c, u, g, unknown or other
  modified_base (148)..(153): a, c, u, g, unknown or other
  modified_base (155)..(155): a, c, u, g, unknown or other
  modified_base (157)..(158): a, c, u, g, unknown or other
  modified_base (161)..(161): a, c, u, g, unknown or other
  modified_base (163)..(163): a, c, u, g, unknown or other
  modified_base (165)..(165): a, c, u, g, unknown or other
  modified_base (168)..(170): a, c, u, g, unknown or other
  modified_base (172)..(172): a, c, u, g, unknown or other
  modified_base (176)..(177): a, c, u, g, unknown or other
  modified_base (179)..(179): a, c, u, g, unknown or other
  modified_base (181)..(182): a, c, u, g, unknown or other
  modified_base (184)..(185): a, c, u, g, unknown or other
  modified_base (187)..(187): a, c, u, g, unknown or other
  modified_base (192)..(195): a, c, u, g, unknown or other
  modified_base (204)..(204): a, c, u, g, unknown or other
  modified_base (206)..(206): a, c, u, g, unknown or other
  modified_base (212)..(213): a, c, u, g, unknown or other
  modified_base (215)..(216): a, c, u, g, unknown or other
  modified_base (222)..(224): a, c, u, g, unknown or other
  modified_base (226)..(228): a, c, u, g, unknown or other
  modified_base (230)..(230): a, c, u, g, unknown or other
  modified_base (232)..(236): a, c, u, g, unknown or other
  modified_base (239)..(240): a, c, u, g, unknown or other
  modified_base (245)..(246): a, c, u, g, unknown or other
  modified_base (250)..(251): a, c, u, g, unknown or other
  modified_base (254)..(254): a, c, u, g, unknown or other
  modified_base (261)..(264): a, c, u, g, unknown or other
gnnnannnnnnnnnnnnnnnnnnnnnnnnnnnnnnnnnnnnnnnnnnnnnnchcnnnnunnnnnnnaccannnnugugauuannnnuuggnnnnnnnnnnnnghgnnnnnnnnnnacnncnnnnnnnnnnnnnnnnnnnnnnagcccnnnnnngngnngcnungnuunnngncucnngnunngnngngccannnnucucaucangnuucuunngnngguggnnnunnngngnnnnngcnncgugnngugnnugnggccccnnnn

SEQ ID NO: 2 - RNA, 92 nt, Artificial Sequence
  Description of Artificial Sequence Synthetic oligonucleotide
  modified_base (1)..(12): a, c, u, g, unknown or other
  modified_base (15)..(19): a, c, u, g, unknown or other
  modified_base (23)..(26): a, c, u, g, unknown or other
  modified_base (28)..(34): a, c, u, g, unknown or other
  modified_base (39)..(42): a, c, u, g, unknown or other
  modified_base (51)..(54): a, c, u, g, unknown or other
  modified_base (59)..(70): a, c, u, g, unknown or other
  modified_base (74)..(83): a, c, u, g, unknown or other
  modified_base (86)..(87): a, c, u, g, unknown or other
  modified_base (89)..(92): a, c, u, g, unknown or other
nnnnnnnnnnnnugnnnnncccnnnnunnnnnnnaccannnnugugauuannnnuggunnnnnnnnnnnngccnnnnnnnnnnacnncnnnn

SEQ ID NO: 3 - RNA, 29 nt, Artificial Sequence
  Description of Artificial Sequence Synthetic oligonucleotide
  modified_base (1)..(10): a, c, u, g, unknown or other
  modified_base (12)..(15): a, c, u, g, unknown or other
  modified_base (17)..(18): a, c, u, g, unknown or other
  modified_base (24)..(29): a, c, u, g, unknown or other
nnnnnnnnnnunnnngnnagcccnnnnnn

SEQ ID NO: 4 - RNA, 19 nt, Artificial Sequence
  Description of Artificial Sequence Synthetic oligonucleotide
  modified_base (1)..(4): a, c, u, g, unknown or other
  modified_base (10)..(11): a, c, u, g, unknown or other
  modified_base (13)..(16): a, c, u, g, unknown or other
  modified_base (18)..(19): a, c, u, g, unknown or other
nnnncccganngnnnnunn

SEQ ID NO: 5 - RNA, 93 nt, Artificial Sequence
  Description of Artificial Sequence Synthetic oligonucleotide
  modified_base (1)..(13): a, c, u, g, unknown or other
  modified_base (16)..(20): a, c, u, g, unknown or other
  modified_base (24)..(27): a, c, u, g, unknown or other
  modified_base (29)..(35): a, c, u, g, unknown or other
  modified_base (40)..(43): a, c, u, g, unknown or other
  modified_base (52)..(55): a, c, u, g, unknown or other
  modified_base (60)..(71): a, c, u, g, unknown or other
  modified_base (75)..(84): a, c, u, g, unknown or other
  modified_base (87)..(88): a, c, u, g, unknown or other
  modified_base (90)..(93): a, c, u, g, unknown or other
nnnnnnnnnnnnnugnnnnnchcnnnnunnnnnnnaccannnnugugauuannnnuggunnnnnnnnnnnnghgnnnnnnnnnnacnncnnnn

SEQ ID NO: 6 - RNA, 94 nt, Artificial Sequence
  Description of Artificial Sequence Synthetic oligonucleotide
  modified_base (1)..(20): a, c, u, g, unknown or other
  modified_base (24)..(27): a, c, u, g, unknown or other
  modified_base (29)..(35): a, c, u, g, unknown or other
  modified_base (40)..(43): a, c, u, g, unknown or other
  modified_base (52)..(55): a, c, u, g, unknown or other
  modified_base (60)..(71): a, c, u, g, unknown or other
  modified_base (75)..(84): a, c, u, g, unknown or other
  modified_base (87)..(88): a, c, u, g, unknown or other
  modified_base (90)..(94): a, c, u, g, unknown or other
nnnnnnnnnnnnnnnnnnnnchcnnnnunnnnnnnaccannnnugugauuannnnuuggnnnnnnnnnnnnghgnnnnnnnnnnacnncnnnnn

SEQ ID NO: 7 - RNA, 92 nt, Artificial Sequence
  Description of Artificial Sequence Synthetic oligonucleotide
  modified_base (1)..(12): a, c, u, g, unknown or other
  modified_base (15)..(19): a, c, u, g, unknown or other
  modified_base (23)..(26): a, c, u, g, unknown or other
  modified_base (28)..(34): a, c, u, g, unknown or other
  modified_base (39)..(42): a, c, u, g, unknown or other
  modified_base (51)..(54): a, c, u, g, unknown or other
  modified_base (59)..(70): a, c, u, g, unknown or other
  modified_base (74)..(83): a, c, u, g, unknown or other
  modified_base (86)..(87): a, c, u, g, unknown or other
  modified_base (89)..(92): a, c, u, g, unknown or other
nnnnnnnnnnnnugnnnnnchcnnnnunnnnnnnaccannnnugugauuannnnuggunnnnnnnnnnnnghgnnnnnnnnnnacnncnnnn

SEQ ID NO: 8 - RNA, 20 nt, Artificial Sequence
  Description of Artificial Sequence Synthetic oligonucleotide
  modified_base (1)..(10): a, c, u, g, unknown or other
  modified_base (12)..(15): a, c, u, g, unknown or other
  modified_base (17)..(18): a, c, u, g, unknown or other
nnnnnnnnnnunnnngnnag

SEQ ID NO: 9 - RNA, 12 nt, Artificial Sequence
  Description of Artificial Sequence Synthetic oligonucleotide
  modified_base (1)..(4): a, c, u, g, unknown or other
  modified_base (10)..(11): a, c, u, g, unknown or other
nnnncccganng

SEQ ID NO: 10 - RNA, 53 nt, Artificial Sequence
  Description of Artificial Sequence Synthetic oligonucleotide
  modified_base (2)..(4): a, c, u, g, unknown or other
  modified_base (6)..(51): a, c, u, g, unknown or other
gnnnannnnnnnnnnnnnnnnnnnnnnnnnnnnnnnnnnnnnnnnnnnnnnch

SEQ ID NO: 11 - RNA, 161 nt, Artificial Sequence
  Description of Artificial Sequence Synthetic polynucleotide
  modified_base (3)..(12): a, c, u, g, unknown or other
  modified_base (15)..(16): a, c, u, g, unknown or other
  modified_base (18)..(39): a, c, u, g, unknown or other
  modified_base (45)..(50): a, c, u, g, unknown or other
  modified_base (52)..(52): a, c, u, g, unknown or other
  modified_base (54)..(55): a, c, u, g, unknown or other
  modified_base (58)..(58): a, c, u, g, unknown or other
  modified_base (60)..(60): a, c, u, g, unknown or other
  modified_base (62)..(62): a, c, u, g, unknown or other
  modified_base (65)..(67): a, c, u, g, unknown or other
  modified_base (69)..(69): a, c, u, g, unknown or other
  modified_base (73)..(74): a, c, u, g, unknown or other
  modified_base (76)..(76): a, c, u, g, unknown or other
  modified_base (78)..(79): a, c, u, g, unknown or other
  modified_base (81)..(82): a, c, u, g, unknown or other
  modified_base (84)..(84): a, c, u, g, unknown or other
  modified_base (89)..(92): a, c, u, g, unknown or other
  modified_base (101)..(101): a, c, u, g, unknown or other
  modified_base (103)..(103): a, c, u, g, unknown or other
  modified_base (109)..(110): a, c, u, g, unknown or other
  modified_base (112)..(113): a, c, u, g, unknown or other
  modified_base (119)..(121): a, c, u, g, unknown or other
  modified_base (123)..(125): a, c, u, g, unknown or other
  modified_base (127)..(127): a, c, u, g, unknown or other
  modified_base (129)..(133): a, c, u, g, unknown or other
  modified_base (136)..(137): a, c, u, g, unknown or other
  modified_base (142)..(143): a, c, u, g, unknown or other
  modified_base (147)..(148): a, c, u, g, unknown or other
  modified_base (151)..(151): a, c, u, g, unknown or other
  modified_base (158)..(161): a, c, u, g, unknown or other
hgnnnnnnnnnnacnncnnnnnnnnnnnnnnnnnnnnnnagcccnnnnnngngnngcnungnuunnngncucnngnunngnngngccannnnucucaucangnuucuunngnngguggnnnunnngngnnnnngcnncgugnngugnnugnggccccnnnn

SEQ ID NO: 12 - RNA, 19 nt, Artificial Sequence
  Description of Artificial Sequence Synthetic oligonucleotide
  modified_base (2)..(5): a, c, u, g, unknown or other
  modified_base (7)..(8): a, c, u, g, unknown or other
  modified_base (14)..(19): a, c, u, g, unknown or other
unnnngnnagcccnnnnnn

SEQ ID NO: 13 - RNA, 44 nt, Artificial Sequence
  Description of Artificial Sequence Synthetic oligonucleotide
  modified_base (3)..(9): a, c, u, g, unknown or other
  modified_base (14)..(17): a, c, u, g, unknown or other
  modified_base (24)..(27): a, c, u, g, unknown or other
  modified_base (32)..(38): a, c, u, g, unknown or other
  modified_base (41)..(41): a, c, u, g, unknown or other
  modified_base (43)..(43): a, c, u, g, unknown or other
ugnnnnnnnccaannnngugauunnnnuuggnnnnnnnccnunc

SEQ ID NO: 14 - RNA, 13 nt, Artificial Sequence
  Description of Artificial Sequence Synthetic oligonucleotide
  modified_base (2)..(5): a, c, u, g, unknown or other
  modified_base (7)..(8): a, c, u, g, unknown or other
unnnngnnagccc